(19) United States
(12) Patent Application Publication
(10) Pub. No.: US 2023/0045506 A1
(43) Pub. Date: Feb. 9, 2023

(54) DRUG INJECTION DEVICE

(71) Applicants: MYO SEONG, Anyang-si (KR); SAMSUNG LIFE PUBLIC WELFARE FOUNDATION, Seoul (KR)

(72) Inventors: Boo Rak LEE, Gimpo-si (KR); Ji Won SEONG, Incheon (KR); Duk Lyul NA, Seoul (KR); Seung Hoon LEE, Seoul (KR); Jeong Min LEE, Seoul (KR); Woo Ram JUNG, Seoul (KR); Kap Soo KIM, Seoul (KR); Nak Soon PARK, Wonju-si (KR)

(73) Assignees: MYO SEONG, Anyang-si (KR); SAMSUNG LIFE PUBLIC WELFARE FOUNDATION, Seoul (KR)

(21) Appl. No.: 17/791,898

(22) PCT Filed: Dec. 21, 2020
(86) PCT No.: PCT/KR2020/018759

§ 371 Date: Jul. 10, 2022

(30) Foreign Application Priority Data
Jan. 10, 2020 (KR) 10-2020-0003713
Jan. 10, 2020 (KR) 10-2020-0003716

(51) Int. Cl. A61M 39/02

(52) U.S. Cl. CPC A61M 39/0247; A61M 2039/025; A61M 2039/027; A61M 2210/0693; A61M 2039/0205; A61M 2039/0282

(57) ABSTRACT

Disclosed is a drug injection device that is fixedly implanted a patient's skull and scalp to inject a drug. The disclosed drug injection device includes a main body that is fixedly implanted in contact with a skull and has an internal hole; a main body fixing part that has an internal hole to be adjacent to the main body and is fixedly coupled to the main body; a movable part that is positioned between the main body and the main body fixing part, has a drug injection hole at a central part thereof, and is able to change its direction to a target point; a sealing part that is configured to seal the drug injection hole of the movable part, into which an injection needle for injecting a drug is inserted; a movable lid part that is positioned above the sealing part to close and fix the movable part, prevents the sealing part from being separated and escaping outside, and identifies an implantation position of the drug injection device to guide a point for repeated administration; and at least two fastening members that fasten an upper part of the main body and an upper part of the main body fixing part to the skull, in which the main body includes a support part that is positioned above the skull for support, an outer diameter of the support part is smaller than an outer diameter of the main body fixing part and is greater than an outer diameter of the movable part, and the main body and the main body fixing part are adjacently coupled to each other to accommodate and fix the movable part in the internal hole of the main body.

### FIELD OF INVENTION

The present invention relates to a drug injection device, and more particularly, to a drug injection device capable of performing drug administration at an accurate position by mounting an intracerebral drug injection device for repeated administration of drugs in the skull of a patient with cranial nerve diseases such as obesity, stroke, spinal nerve, or dementia, in a long term.

### BACKGROUND OF THE INVENTION

In recent years, diseases that lower quality of life, in particular, brain-related diseases that increase social and economic costs have become a lot of social issues. The brain-related diseases, for example, degenerative brain diseases, mental disorders, brain tumors and strokes, obesity, spinal nerves, or the like tend to increase year by year. For patients with terminal cancer and chronic pain with brain diseases, it is necessary to relieve pain through continuous and repeated administration or taking of analgesics. For dementia patients suffering from degenerative brain diseases such as Alzheimer's disease or psychiatric diseases such as Parkinson's disease, it is necessary to continuously and repeatedly administer therapeutic agents such as chemical drugs or stem cells to maximize a therapeutic effect.

Currently, in order to increase the therapeutic effect of the brain-related diseases, treatment methods using repeated oral and intravenous administration over a continuous period have been widely used. However, the existing methods have limitations in that it may be difficult to take drugs in a case where the patient's digestive system is weakened, and a variety of side effects may be induced due to systemic circulation of therapeutic agents. Further, the physical pain and economic burden of the patient according to inpatient treatment and administration of the therapeutic agent is large.

On the other hand, a method of directly administering a drug to the brain has an excellent effect compared to the existing methods, but there are few side effects due to systemic circulation of the drug. In the intracerebral direct administration method, for example, in the case of a patient with dementia, a therapeutic agent such as a chemical drug or stem cells may be directly administered to a target point, which is the hippocampus, for treatment. However, there is a difficulty in that the drug targeting the brain should pass through the blood-brain barrier (BBB). Further, in order to increase the therapeutic effect, it is necessary to repeatedly administer the drug to an accurate target point.

Accordingly, in the related art, drug injection devices that can be implanted in the brain for a long period of time and can perform repeated administration to an accurate target point have been developed. However, since a guide tube is changed in position or is inserted only in one direction, it is difficult to re-inject a drug to an accurate position, which lowers the accuracy of the procedure. In particular, in repeatedly administering the drug to the same lesion in the brain parenchyma, the accuracy of the guide tube is lowered, so that the drug injection device needs to be repeatedly implanted, which leads in the patient's economic burden and unsatisfactory treatment effects.

### SUMMARY OF THE INVENTION

In order to solve the above problems, an object of the present invention is to provide a drug injection device that is able to directly inject a drug into the brain for treating brain diseases such as dementia, obesity, stroke, or spinal nerves, precisely and accurately positions a guide tube to be close to a target point as a lesion, and enables direction change with reference to a central axis.

Another object of the present invention is to provide a drug injection device that can easily identify an implantation site by incising the implantation site without surgery through general anesthesia and can repeatedly administer a drug to the site.

Still another object of the present invention is to provide a drug injection device that prevents a reverse flow of the drug and intrusion of contaminants from the outside according to repeated administration by doubly blocking the outside of the skull and the inside of the brain parenchyma with a sealing part and a movable lid part.

According to an aspect of the present invention, there is provided a drug injection device that is fixedly implanted between the skull and the scalp of a patient to inject a drug, including: a main body that is fixedly implanted in contact with the skull and has an movable part accommodating hole as an internal hole; a main body fixing part that has an internal hole to be adjacent to the main body and is fixedly coupled to the main body to face the main body; a movable part that is positioned between the main body and the main body fixing part, has a drug injection hole at a central part thereof, and is able to change its direction to a target point; a sealing part that is configured to seal the drug injection hole of the movable part, into which an injection needle for injecting a drug is inserted; a movable lid part that is positioned above the sealing part to close and fix the movable part, prevents the sealing part from being separated and escaping from the outside, and identifies an implantation position of the drug injection device to guide a point for repeated administration; and at least two fastening members that fasten an upper part of the main body and an upper part of the main body fixing part to the skull, in which the main body includes a support part that is positioned above the skull for support, an outer diameter of the support part is smaller than an outer diameter of the main body fixing part and is greater than an outer diameter of the movable part, and the main body and the main body fixing part are adjacently coupled to each other to accommodate and fix the movable part in the internal hole of the main body.

The drug injection device may further include a guide tube that is connected to the movable part, is inserted into the brain parenchyma, and is inserted to a position close to the target point, and guides the injection needle for injecting the drug into the target point in the direction of the target point.

The guide tube may further include a tube connection part that is mounted on a lower end part of the drug injection hole, connects the movable part and the guide tube, and is formed with a hole connected to the inside of the guide tube.

The support part may include at least one first fastening hole that is positioned at the upper part of the main body, in which the fastening member is fixedly inserted; and at least one fixing protrusion that is fixedly coupled to the main body fixing part.

The main body fixing part may include an identification part that has a curved surface that protrudes upwards to be able to identify the implantation position of the drug injection device; a fixing hole formed at a central part thereof for fixing the outside of the movable part so that the movable part is not moved; at least one second fastening hole formed at an edge side thereof, in which the fastening member is fixedly inserted from above; a coupling groove that has a curved lower part that is concave in shape and matches the shape of the support part of the main body for easy coupling; and at least one fixing groove to be coupled to the fixing protrusion inside the coupling groove.

The movable part may include a movable main body part that is positioned in the movable part accommodating hole, and is formed with a curved surface in shape to enable direction change, and the movable main body part is formed with a drug injection hole for guiding the drug to be injected to the target point at a central part thereof.

The drug injection hole may include a first accommodating part that accommodates the sealing part located in the internal hole of the movable part, and a second accommodating part that is connected to the first accommodating part, is located at a lower end part of the inside of the movable part, and accommodates the injection needle and the guide tube inserted during drug injection.

The first accommodating part may be formed with a screw-thread from the start of the internal hole of the movable part to a point at which the sealing part is accommodated.

The movable part may be formed in a spherical shape having a sphericity less than 0.03 mm.

The sealing part may be made of a silicone material.

The movable lid part may include a lid upper end part that is formed with an injection needle accommodating part as a hole that is inclined downward, into which the injection needle is injected, at a central part thereof, includes the needle accommodating part therein, and is positioned outside the movable part, and a lid fastening part that is connected to the lid upper end part, is formed with a screw-thread on the outside thereof, and is screw-coupled with the drug injection hole.

The fastening member may include a body part that fixedly implants at least one of the main body and the main body fixing part in the skull, and a head part that is connected to the body part, and a head part may include a horizontal groove that is positioned at a central part thereof to be horizontally coupled to a fastening tool in fastening, and an identification groove that is formed to be identifiable in a cross shape with reference to the horizontal groove.

The body part may be fixedly implanted in the skull, may be formed with a tapered screw-thread to the end thereof, and may be formed with at least one pressure reducing groove.

The guide tube may be configured so that at least one drug injection hole is formed on one side or both sides of the guide tube so that the drug flows without accumulating, and the inner and outer diameters of the tip of the guide tube are formed in a curved shape to minimize tissue damage when inserted into the skull.

In the drug injection device, at least one of the main body, the main body fixing part, the movable part, the movable lid part, and the guide tube may be made of polyether ether ketone.

The fastening member may be made of a titanium material.

The sealing part may further include a filter for preventing introduction of foreign substances, and may include an integrated sealing filter membrane that is easy to replace and install at a time by integrating the filter and the sealing part.

The guide tube may be configured so that a maximum angle by rotation for the target point to the left and right from the central axis with reference to a plane in contact with the skull is within 60°.

Since the drug injection device according to the present invention can easily identify the implantation site by incising the implantation site without surgery through general anesthesia and can repeatedly administer the drug in the site, it is possible to save economic burden and time, and to increase treatment effects through repeated administration.

Further, since the drug injection device according to the present invention can rotate the movable part capable of changing the direction and the guide tube inserted therein in the direction of the target point, which is a lesion, and can insert the injection needle through the guide tube to accurately administer the drug to the target point, it is possible to improve the precision and accuracy of drug administration.

In addition, since the drug injection device according to the present invention can prevent the reverse flow of the drug and intrusion of contaminants from the outside according to the repeated administration by doubly blocking the outside of the skull and the inside of the brain parenchyma with the sealing part and the movable lid part, it is possible to reduce side effects due to infection.

Furthermore, since the drug injection device according to the present invention is made of a biocompatible material that does not adhere to brain tissues after implantation to have high implant stability, the patient can perform daily activities without immune rejection even after long-term implantation.

### BRIEF DESCRIPTION OF DRAWINGS

FIG. 1 is an assembled perspective view schematically showing a drug injection device according to an embodiment of the present invention.

FIG. 2 is a view showing a state where the drug injection device in FIG. 1 is mounted by finding a position of a target point in the skull of a patient using a stereotaxic guide mechanism.

FIG. 3 is a cross-sectional view showing a state where a drug is injected after the drug injection device in FIG. 2 is mounted.

FIG. 4 is a side sectional view schematically showing a state where the drug injection device of FIG. 3 can be operated after being fully assembled.

FIGS. 5(a) to 5(c) are a plan view, a side view, and a side sectional view showing a main body of the drug injection device according to the embodiment of the present invention.

FIGS. 6(a) to 6(d) are a plan view, a bottom view, a c-c′ sectional view, and a d-d′ sectional view showing a main body fixing part of the drug injection device according to the embodiment of the present invention.

FIGS. 7(a) and 7(b) are a side view and a side sectional view showing a movable part of the drug injection device according to the embodiment of the present invention.

FIGS. 8(a) to 8(c) are a plan view, a side view, and a side sectional view showing a movable lid part of the drug injection device according to the embodiment of the present invention.

FIGS. 9(a) and 9(b) are a plan view and a side view showing a fastening member of the drug injection device according to the embodiment of the present invention.

FIGS. 10(a) to 10(c) are a plan view, a side view, and a side sectional view showing a guide tube of the drug injection device according to the embodiment of the present invention.

FIGS. 11(a) and 11(b) are a perspective view and a side sectional view showing a first modified example of a sealing part of the drug injection device according to the embodiment of the present invention.

FIG. 12 is a side sectional view showing a second modified example in which a filter is integrated into the sealing part of the drug injection device according to the embodiment of the present invention.

FIG. 13 is a side sectional view showing a state where an end part of the guide tube of the drug injection device according to the embodiment of the present invention is deformed.

FIG. 14 is a side sectional view showing a drug injection device according to a second embodiment of the present invention.

FIG. 15 is a side sectional view showing a drug injection device according to a third embodiment of the present invention.

FIG. 16 is a front view showing a drug injection device according to a fourth embodiment of the present invention.

### DETAILED DESCRIPTION OF THE INVENTION

Hereinafter, a drug injection device according to an embodiment of the present invention will be described in detail with reference to the accompanying drawings. The accompanying drawings may be shown by exaggerating or simplifying a part of a configuration for convenience and clarity of explanation and understanding of the configuration and operation of the invention, and each component may not exactly match an actual size.

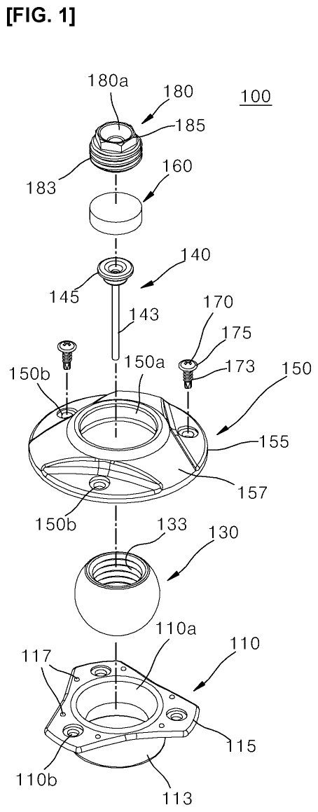

FIG. 1 is an assembled perspective view schematically showing a drug injection device according to an embodiment of the present invention.

Referring to FIG. 1, a drug injection device 100 may be fixedly implanted between a skull 1 and a scalp 3 of a patient P to inject a drug. The drug injection device 100 may include a main body 110, a main body fixing part 150, a movable part 130, a sealing part 160, a movable lid part 180, and a fastening member 170.

The main body 110 may be fixedly implanted in contact with the skull 1, in which an internal hole may be formed. Further, the main body 110 may be coupled to the main body fixing part 150 to be adjacent to each other, and the movable part 130 may be fixedly accommodated in the internal hole of the main body 110. The main body 110 may include a support part 115, an insertion part 113, and a movable part accommodating hole 110a. The main body 110 may include the support part 115 that is positioned above the skull 1 for support, and an outer diameter of the support part 115 may be smaller than an outer diameter of the main body fixing part 150 and may be greater than an outer diameter of the movable part 130. The support part 115 may be coupled to the main body fixing part 150 to be adjacent to each other so that the movable part 130 is fixedly accommodated in the internal hole of the main body 110. The support part 115 is positioned above the main body 110, and may include at least one first fastening hole 110b in which the fastening member 170 is fixedly inserted and at least one fixing protrusion 117 that is fixedly coupled to the main body fixing part 150. The insertion part 113 is inserted into the skull 1, and may be located in contact with the skull 1. The movable part accommodating hole 110a may accommodate the movable part 130 inside the main body 110.

The main body fixing part 150 may have an internal hole to be adjacent to the main body 110, and may be fixedly coupled to the main body 110. Further, the main body fixing part 150 may include an identification part 157, a fixing hole 150a, a second fastening hole 150b, and a support coupling groove 157a. The identification part 157 has an upper part that protrudes in the form of a curved surface, so that an implantation position of the drug injection device 100 can be identified. The fixing hole 150a may fix an outer surface of the movable part 130 so that the movable part 130 does not move at a central part thereof. At least one second fastening hole 150b is formed at an edge side of the main body fixing part 150 so that the fastening member 170 can be fixedly inserted thereinto from above. A support coupling groove 157a may be formed as a concave groove on a surface that is in contact with the support part 115 to match the support part 115, so as to be easily coupled to the support part 115. Further, the support coupling groove 157a may further form at least one of a head accommodating groove 157b and a projection accommodating groove 157c. The head accommodating groove 157b accommodates a protrusion of a head part 175 of the fastening member 170 coupled to the support part 115, so that in a case where the main body 110 is coupled with the main body fixing part 150, it is possible to reduce friction between contacting surfaces of the support part 115 and a mounting part 155. In addition, the head accommodating groove 157b can prevent reduction of a fastening force with respect to the main body fixing part 150 due to interference of the head part 175 of the fastening member 170 fastened to the main body 110, and can be easily and suitably coupled to the main body fixing part 150. At least one protrusion accommodating groove 157c is formed in one support coupling groove 157a, is positioned to face the fixing protrusion 117 of the support part 115, and may accommodate the fixing protrusion 117 to be coupled thereto.

Accordingly, the main body fixing part 150 can provide coupling forces in at least three points including the second fastening hole 150b, the support coupling groove 157a, and the protrusion accommodating groove 157c when coupled to the main body 110, and thus, even in the case of a long-term implantation on the skull 1, the main body fixing part 150 and the main body 110 are not separated from each other, to thereby improve the fixing force.

The movable part 130 is positioned between the main body 110 and the main body fixing part 150, and a drug injection hole 133 is formed at a central part thereof to enable direction change toward a target point 7. The movable part 130 is positioned in the movable part accommodating hole 110a, and includes a movable main body 131 that is formed in a curved outer surface and can change its direction, in which a drug injection hole 133 through which a drug is injected to the target point 7 is formed inside the movable body 131. The movable part 130 may be formed in a spherical shape with a roundness less than 0.03 mm. In a case where the roundness exceeds 0.03 mm, a surface thereof in contact with the movable part accommodating hole 110a of the main body 110 may be uneven, so that a desired rotation in the direction of the target point 7 may be difficult. Further, a surface roughness (Ra) of the movable part 130 may be greater than 0.1 μm and smaller than 0.5 μm. In a case where the surface roughness is equal to or less than 0.1 μm, the roundness may deviate from the limit to lower the fixing force, and in a case where the surface roughness is equal to or greater than 0.5 μm, the surface may become rough to cause friction in contact with the main body 110, thereby weakening the fixing force. That is, it is difficult to secure a complete rotation in adjusting the angle. In addition, a defect rate in assembling the drug injection device 100 may become high.

The drug injection hole 133 may include a first accommodating part 133a and a second accommodating part 133b. The first accommodating part 133a may accommodate the sealing part 160 positioned in the internal hole of the movable part 130. The second accommodating part 133b is located at a lower end part of the inside of the movable part 130 to be connected to the first accommodating part 133a, and accommodates an injection needle 191 and a guide tube 140 inserted during drug injection. The first accommodating part 133a may be formed with a screw-thread from the start of the internal hole of the movable part 130 to the point at which the sealing part 160 is accommodated. The sealing part 160 is provided to seal the drug injection hole 133 of the movable part 130, in which the injection needle 191 for injecting the drug may be inserted. The sealing part 160 is made of silicone, but is not limited thereto, and may be made of at least one of other synthetic resin materials. The sealing part 160 may be restored to its original shape again even in a case where the injection needle 191 is inserted. Accordingly, the sealing part 160 is inserted into the movable part 130 to block contaminants from the outside to thereby prevent infection after implantation of the drug injection device 100.

The movable lid part 180 is positioned above the sealing part 160 to fixedly close the movable part 130 to thereby prevent the sealing part 160 from being separated from the outside. The movable lid part 180 may include a needle accommodating part 180a, a position indicating part 185, and a movable fastening part 183. The needle accommodating part 180a may have a hole inclined downward so that the injection needle 191 is injected at a central part thereof. The position indicating part 185 may be configured so that the needle accommodating part 180a is positioned in the inside thereof, and may be located outside the movable part 130. The movable fastening part 183 is connected to the position indicating part 185, and is formed with a screw-thread on the outside thereof to be screw-coupled with the drug injection hole 133.

Referring to FIG. 1, the drug injection device 100 may further include the guide tube 140. The guide tube 140 connected to the movable part 130 may be inserted to a position close to the target point 7 inside a brain parenchyma 5, and may guide the injection needle 191 through which a drug is injected in the direction of the target point 7. The guide tube 140 coupled to the movable part 130 may be inserted in the direction of the target point 7 from a central axis CL with reference to the point in contact with the skull 1. The guide tube 140 may include a tube connection part 145 that is mounted at a lower end part of the drug injection hole 133 to connect the movable part 130 and the guide tube 140. The tube connection part 145 may be formed with a hole connected to the inside of the guide tube 140.

In the drug injection device 100 according to the present embodiment, at least one of the main body 110, the main body fixing part 150, the movable part 130, the movable lid part 180, and the guide tube 140 may be made of polyether ether ketone (PEEK), but the present invention is not limited thereto, and various materials having implantation bonding properties may be used.

The fastening member 170 may include at least two members that fasten an upper part of the main body 110 and an upper part of the main body fixing part 150 to the skull 1. The fastening member 170 may include a body part 173 and a head part 175. The body part 173 may fixedly implant at least one of the main body 110 and the main body fixing part 150 in the skull 1. The body part 173 is fixedly implanted in the skull 1, has a tapered screw-thread to the end part thereof, and may be formed with at least one pressure reducing groove 173a. The head part 175 is connected to the body part 173, and may include a horizontal groove 175b formed at a central part thereof to be horizontally fastened to a fastening tool in fastening and an identification groove 175a formed to be identifiable in a cross shape with reference to the horizontal groove 175b. Further, the fastening member 170 may be made of a titanium material, but is not limited thereto, and various materials having implantation bonding properties and implant stability may be used.

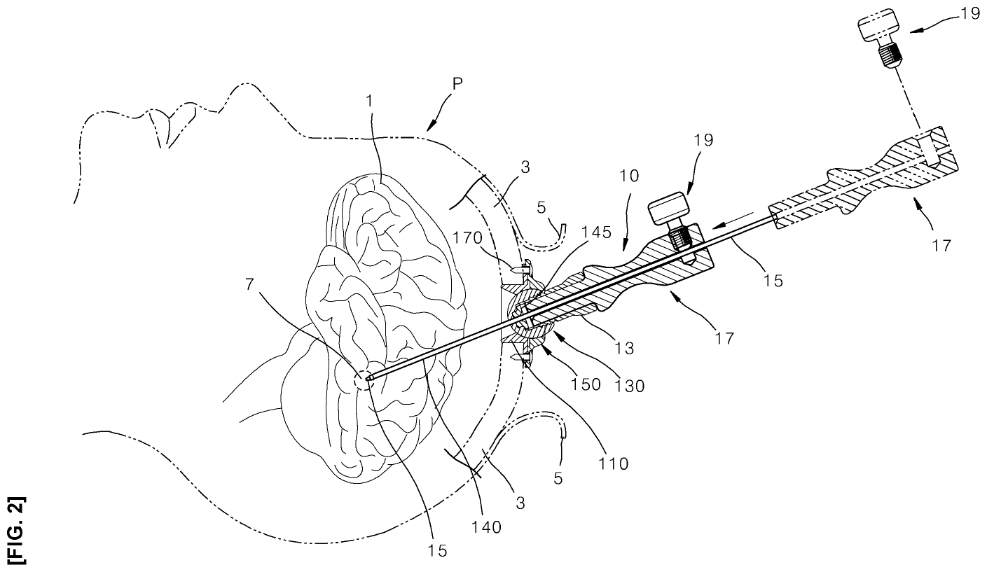

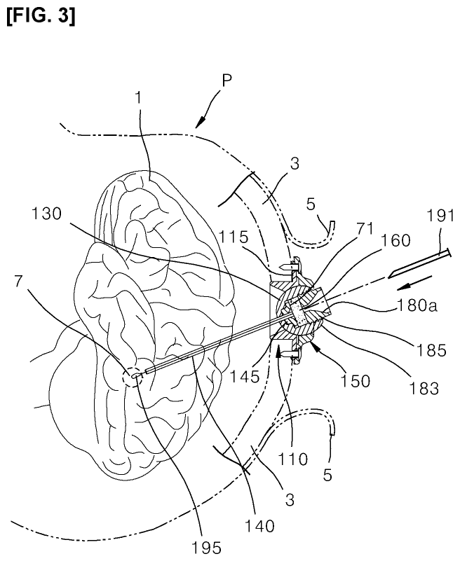

FIG. 2 is a view showing a state where the drug injection device in FIG. 1 is mounted by finding a position of a target point in the skull of a patient using a stereotaxic guide mechanism, and FIG. 3 is a cross-sectional view showing a state where a drug is injected after the drug injection device in FIG. 2 is mounted.

Referring to FIGS. 2 and 3, in performing a procedure on a patient P related to a brain disease requiring drug injection into the brain, insertion of the drug injection device 100 may be guided using a stereotaxic guide device 10. Here, the brain disease may be related to, for example, brain tumor, stroke, a dementia disease such as Alzheimer's, and a mental disease such as Parkinson's, depression, or schizophrenia. In order to implant the drug injection device 100 by making a burr-hole in the skull 3 of the patient P, a ‘U’-shaped incision may be formed at a position of the temporal bone, parietal bone, or the like of the skull 3. Here, the drug injection device 100 includes the main body 110, the movable part 130, the guide tube 140, the main body fixing part 150, the sealing part 160, and the movable lid part 180, as described above. The main body 110 is implanted in the skull 3, and an upper end part of the main body 110 is located between the scalp 5 and the skull 3, and a lower end part of the main body 110 is inserted into the skull 3 to be positioned therein. The movable part 130 having a spherical shape with an open upper part is inserted into the main body 110, and may perform direction change toward a target point 7 that is a lesion in the brain parenchyma.

Referring to FIG. 2, the stereotaxic guide device 10 is coupled to the movable part 130 to change the direction of the guide tube 140 located on the central axis CL from the center CL to thereby guide the guide tube 140 to be located at the target point 7. Here, the stereotaxic guide device 10 may include a guide handle 13, a position adjusting part 15, a position adjusting handle part 17, and a bolt part 19. Specifically, the guide handle 13 is mounted to the movable part 130, and the guide tube 140 is coupled to the position adjusting part 15 of a long rod shape to be inserted inside the movable part 130. In order to prevent the position adjusting part 15 from shaking or breaking during insertion, the position adjusting handle part 17 may be coupled to an end part of the position adjusting part 15. The stereotaxic guide device 10 may be removed from the skull 3 after positioning the guide tube 140 as close as possible to the target point 7.

On the other hand, FIG. 2 shows a state in which the guide tube 140 is positioned at the target point 7 using the stereotaxic guide device 10. The present invention is not limited thereto, and although not shown in the drawings, the stereotaxic guide device 10 may further include a connection guide part (not shown). The connection guide part may guide the guide tube 140 to be accurately inserted into the guide handle 13. The connection guide part having a pipe shape includes at least a pair of detachable parts, and may be additionally mounted on an upper end part of the guide handle 13. Further, an end part of the connection guide part is inserted into the guide handle 13 and the other end part thereof is mounted on the guide handle 13 to further guide the insertion of the guide tube 140. The connection guide part may be separated and removed in a state of coming into contact with the tube connection part 145 to be inserted. Here, as shown in FIG. 2, the leading end part of the position adjusting handle part 17 may push the tube connection part 145 to be positioned at the lower end part of the movable part 130.

As shown in FIG. 3, the injection needle 191 for injecting brain disease-related drugs therein may be inserted into the guide tube 140. Here, the injection needle 191 may accommodate, for example, a trocar needle, a needle for inspection, or the like therein. However, the invention is not limited thereto, various surgical instruments and equipment, for example, navigation probes, stimulation leads, ablation probes or catheters, injection or fluid delivery devices, biopsy needles, extraction tools, etc. may be inserted to perform diagnostic and/or therapeutic procedures.

Further, various brain disease-related drugs may be injected using the drug injection device 100. For example, the above-mentioned drugs may include dementia-related chemicals such as amyloid hypothesis (substance that inhibits β-amyloid protein production), Aβ protein aggregation inhibitor, tau aggregation inhibitor, cholinease inhibitor, NMDA receptor or antagonist, cholinergic precursor, antioxidant, diabetes treatment drugs; stem cells such as human umbilical cord blood cells, human umbilical cord blood-derived mesenchymal stem cells, neural stem cells, or bone marrow stem cells; drugs for treating psychiatric diseases such as Parkinson's, depression, or schizophrenia; brain tumor and stroke-related therapeutic drugs; and the like.

Referring to FIGS. 2 and 3, a method for mounting and operating the drug injection device 100 according to the embodiment of the present invention as follows will be described in detail.

<Mounting and Operating Method>

A pre-operative preparation step is performed by finding a position of the target point 7 to mark the position on the skull 1 on coordinates using a navigation device (not shown) and MRI imaging (S10).

The pre-operative preparation step (S10) may include a step of inputting the position using a navigation probe and transmitting images and photos by an MRI machine (S11).

Further, the pre-operative preparation step (S10) may include a step of finding the position of the target point 7 in the brain of the patient P on the basis of images or photos obtained by MRI, and marking a plurality of reference sections to the scalp 3, that is, the subcutaneous layer (S13).

The type of the navigation probe may be classified into a measurement probe and a verification probe. After checking a path before grating using the verification probe, the marking may be performed on the scalp 3 of the patient P. The pre-operative preparation step (S10) may include a step of incising the marked scalp 3 of the patient P in the shape of ‘L’, ‘¬’, ‘S’, or the like, for example (S15).

The pre-operative preparation step (S10) may include a step of spreading the incised site to secure a space for perforation after the incision, and tying the incised subcutaneous layer 5 with a thread for treatment to fix the site so as not to be spread during the treatment (S17). Here, the fixing and spreading may be performed using forceps or the like. Further, the pre-operative preparation step (S10) may include a step of inhaling foreign substances, blood, etc. using a suction catheter together with physiological saline as necessary in order to secure sight and visibility during the treatment (S17a).

The pre-operative preparation step (S10) may include a step of removing bone debris or residues with tweezers 1 after making a burr hole in the skull 1 that is incised and exposed (S19).

Then, a step of implanting the drug injection device 100 in the perforated site obtained after incising the scalp 5 of the patient P and making the burr hole in the skull 1 may be performed (S20).

First, a step of fixedly implanting and fastening the main body 110 in the skull 1, inserting the movable part 130 into the movable part accommodating hole 110a, and mounting at least two fastening members 170 in the first fastening hole 110b may be performed (S30).

The main body fixing part 150 may be mounted above the main body 110 to face the main body 110 with the movable part 130 being interposed therebetween. Further, the fixing hole 150a formed in the main body fixing part 150 and the movable part accommodating hole 110a of the main body 110 may be mounted to communicate with each other while accommodating the movable part 130 (S40).

Then, at least two fastening members 170 are mounted in the second fastening hole 150b formed in the main body fixing part 150. Here, a step of fixing the fastening member 170 such that the movable part 130 is movable and mounting the guide handle 13 to the movable part 130 may be performed (S50).

A step of mounting a probe accommodating part to which an optical guide part is assembled inside the guide handle (13), inserting a navigation probe therein, and performing adjustment for finding the target point 7 while performing direction change may be performed (S60).

After the accurate target point 7 is set, the navigation probe (not shown) and the probe accommodating part (not shown) may be separated and removed from the guide handle 13 (S70).

The guide tube 140 that includes the tube connection part 145 is prepared. Then, the position adjusting handle 17 and the bolt part 19 are coupled to the position adjusting part 15 as one set. Here, in a case where the position adjusting handle part 17 reaches the portion in contact with the tube connection part 145, the bolt part 19 may be fixedly fastened to the fastening accommodating part 179 of the position adjusting handle part 17 (S80).

Here, a pair of detachable connection guide parts (not shown) may be mounted at an upper end part of the guide handle 13, to thereby further guide the insertion of the guide tube 140 including the position adjusting part 15 in a central part thereof. After the tube connection part 145 comes into contact with the connection guide parts (not shown), a step of separating and removing the connection guide parts (not shown) and inserting the position adjusting handle part 17 until the leading end part of the position adjusting handle part 17 pushes the tube connection part 145 to be positioned at the lower end part of the movable part 130 is performed (S90).

The position adjusting handle part 17 may be adjusted so that the position adjusting part 15 is positioned at the end part of the guide tube 140 and is moved together to rotate in the direction of the target point 7 in the brain parenchyma 5, and reaches the target point 7 before the guide tube 140 (S100).

In a case where the guide tube 140 is positioned close to the target point 7, the position adjusting part 15 is separated and removed from the guide handle 13, and the guide handle 13 is finally separated and removed from the movable part 130 (S110).

On the other hand, for the rotation in the direction of the target point 7 in the brain parenchyma 5, the method of positioning the guide tube 140 using the position adjusting handle part 17 included in steps S50 to S110 is not limiting, but a method of rotating only the movable part 130 into which the guide tube 140 is inserted within a maximum angle of 60° to be positioned toward the target point 7, without steps S50 to S110, may be used.

The sealing part 160 is inserted into the inside of the movable part 130 rotated in the direction of the target point 7 in the brain parenchyma 5, and the movable lid part 180 may be mounted thereon using a dedicated driver (not shown). Further, the drug injection device 100 may be fixed on the skull 1 of the patient P by fastening the fastening member 170 loosely located at the upper part of the main body 110 once again (S120).

After the initial implantation of the drug injection device 100 is completed, the position indicating part 185 of the movable lid part 180 may be visually identified to find the position (S130).

The injection needle 191 including a therapeutic agent 195 is sequentially inserted along the needle accommodating part 180a of the movable lid part 180, the sealing part 160, the insertion guide hole 145a of the tube connection part 145, and the guide tube 140 to reach the target point 7, to thereby repeatedly administer the therapeutic agent 195 over a long period of time (S140).

Specifically, the drug injection step (S140) may include a step of causing the injection needle 191 containing the drug of the therapeutic agent 195 to pass through the movable lid part 180, the needle accommodating part 180a, the sealing part 160, the insertion guide hole 145a and the guide tube 140 to reach the final target point 7, and administering the drug (S145). Here, each of the insertion guide hole 145a and the needle accommodating part 180a has a shape that becomes narrow toward the bottom like a funnel, so that the drug can be easily introduced into the guide tube 140. Further, the drug injection step (S140) may include a step of repeatedly administering the drug a plurality of times over a long period of time in a state where the injection needle 191 containing the drug of the therapeutic agent 195 reaches the final target point (S147). Here, at least one drug injection hole 140a may be formed at an end part of the guide tube 140 so that the drug is evenly distributed to the target point 7 without accumulation at the end part of the guide tube 140 (S149).

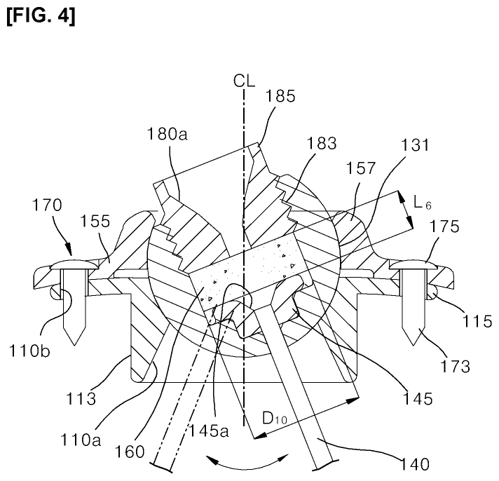

FIG. 4 is a side sectional view schematically showing a state where the drug injection device of FIG. 3 can be operated after being fully assembled.

Referring to FIG. 4, the drug injection device 100 according to the present embodiment includes the main body 110, the movable part 130, the main body fixing part 150, the guide tube 140, the sealing part 160, and the movable lid part 180 to be fully assembled. The main body 110 may include the insertion part 113 to be inserted into the skull 1 at a lower end part thereof, and the support part 115 for horizontally supporting the upper part of the skull 1 at an upper end part thereof. The mounting part 155 of the main body fixing part 150 may be mounted in contact with the support part 115. Here, in order to further increase the coupling and fixing force between the main body 110 and the main body fixing part 150, the fastening member 170 may be further mounted therebetween. Specifically, the second fastening hole 150b is formed at one end part of the support part 115, and the fastening member 170 may be mounted in the second fastening hole 150b. Further, the other end part of the support part 115 is formed with the head accommodating groove 157b that faces and contacts the first fastening hole 110b of the main body 110, and the head part 175 of the fastening member 170 fastened to the first fastening hole 110b may be seated therein. Accordingly, in the drug injection device 100, the first fastening hole 110b and the second fastening hole 150b are formed at at least three different points. That is, the fastening member 170 may be mounted at at least six points when the main body 110 and the main body fixing part 150 are fastened. Accordingly, after the drug injection device 100 is implanted, the main body 110 and the main body fixing unit 150 cannot be easily separated and detached from each other, thereby maintaining the initial fixing force.

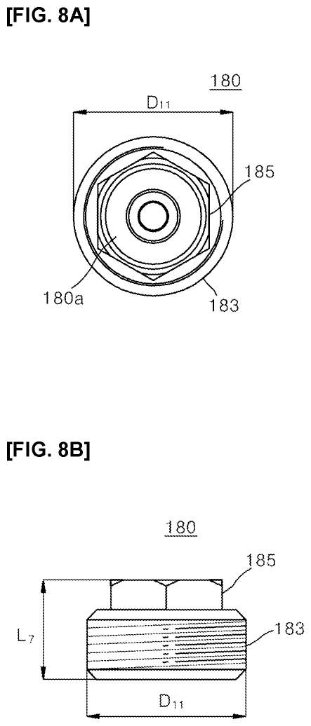

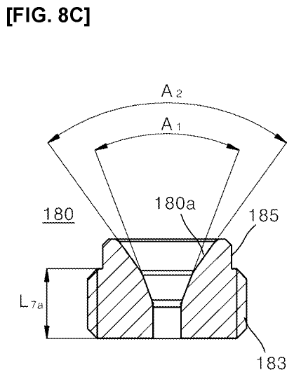

Further, the movable main body 131 that has the curved outer surface can change its direction may be mounted in the movable part accommodating hole 110a formed at the central part of the main body (110). The drug injection hole 133 may be formed in the movable body 131 to guide the drug to be injected to the target point 7. The guide tube 140 is mounted at the lower end part of the drug injection hole 133 and is rotatable in the direction of the target point 7. Here, the guide tube 140 coupled with the movable part 130 may have a maximum angle within 60° as a rotatable range in the direction of the target point 7 on the left and right from the central axis CL with reference to the surface thereof in contact with the skull 1. In a case where the maximum angle exceeds 60°, an excessive load is generated on one side of the mounting part 155 coupled to the support part 115, so that the coupling balance is broken, which may result in separation of the mounting part 155. Further, in a case where the position of the guide tube 140 mounted in the movable part 130 is determined in the direction of the target point 7, the tube connection part 145 is seated in the drug injection hole 133, and the sealing part 160 is mounted at the upper part of the tube connection part 145. Here, the sealing part 160 may have a predetermined height L₆ and an outer diameter D₁₀ to be inserted into the movable part 130. A condition range of the sealing part 160 will be described later with reference to FIG. 8. The movable fastening member 170 of the movable lid part 180 may be mounted above the sealing part 160. The movable lid part 180 may have the needle accommodating part 180a that is hollow at a central part thereof. Further, the position indicating part 185 of the movable lid part 180 may be formed in a hexagonal shape to indicate the implanted position. Accordingly, in a case where the drug injection device 100 is implanted in the skull 1 to cover and suture the scalp 3, the implanted position can be found through the position indicating part 185, and the injection needle 191 can be sequentially inserted into the needle accommodating part 180a, the sealing part 160, the insertion guide hole 145a of the tube connection part 145, and the guide tube 140.

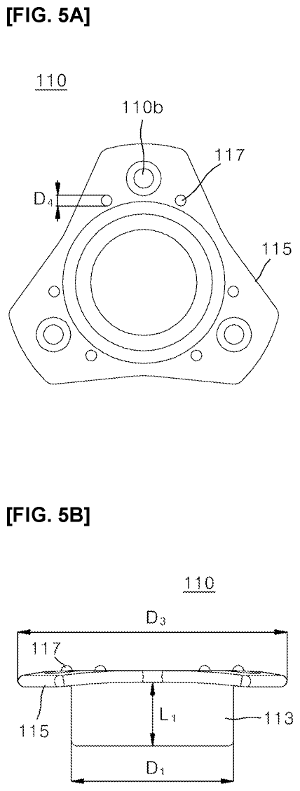

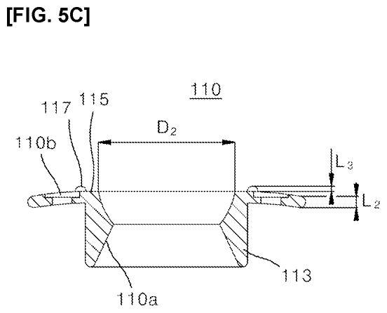

FIGS. 5(a) to 5(c) are a plan view, a side view, and a side sectional view showing a main body of the drug injection device according to the embodiment of the present invention.

Referring to FIGS. 5(a) to 5(c), the main body 110 may have the movable part accommodating hole 110a that accommodates the movable part 130 therein. Further, the main body 110 is located above the skull 1, and may include the support part 115 that fixedly supports the skull 1 of the patient P and the insertion part 113 to be inserted into the skull 1.

Here, the main body 110 may satisfy at least one of Expressions 1 and 2.

1≤D₁/D₂≤3 [Expression 1]

2≤L₁≤7 [mm] [Expression 2]

Here, D₁ represents an outer diameter of the insertion part 113, D₂ represents an inner diameter of the movable part accommodating hole 110a, and L₁ represents an insertion height of the insertion part 113.

In a case where D₁/D₂ is smaller than 1, since the thickness of the main body 110 becomes excessively thin, it may be difficult to fix the movable part 130, and in a case where D₁/D₂ is greater than 3, since the movable part 130 may not be seated to be exposed outside, the risk of infection increases. Accordingly, in a case where the condition range of Expression 1 is satisfied, the main body 110 can be positioned between the skull 1 and the scalp 3, and can have an appropriate thickness with which the movable part 130 is seated in the movable part accommodating hole 110a.

On the other hand, in a case where L₁ is smaller than 2, since the insertion part 113 has a height lower than the thickness of the skull 1, it is not possible to secure a space for accommodating the movable part 130, and in a case where L₁ is greater than 7, since the main body 110 may have a thickness of the insertion part 113 that presses the brain parenchyma 5 beyond the thickness of the skull 1, there is a concern that side effects such as headache and dizziness occur to the patient P after the procedure. Accordingly, in a case where the condition range of Expression 2 is satisfied, the insertion part 113 can be inserted into the skull 1 to have an appropriate height that does not press the brain parenchyma 5.

The support part 115 may include the first fastening hole 110b and the fixing protrusion 117. At least one fastening hole 110b may be positioned at an upper part of the main body 110, and the fastening member 170 may be fixedly inserted thereinto. At least one fixing protrusion 117 may be provided to be fixedly coupled to the main body fixing part 150.

At least one fixing protrusion 117 and the first fastening hole 110b may be formed to satisfy Expression 3.

12≤D₃/L₂≤60 [Expression 3]

Here, D₃ represents an outer diameter of the support part 115, and L₂ represents a height of the support part 115.

In a case where D₃/L₂ is smaller than 12, since the height of the support part 115 is high compared to the outer diameter, stable seating on the skull 1 may be difficult, and in a case where D₃/L₂ is greater than 60, since the outer diameter of the support part 115 becomes excessively large, the distance between the first fastening holes 110b becomes large, and thus, the fixing force may be weakened as time passes. Accordingly, in a case where the condition range of Expression 3 is satisfied, the support part 115 can have a diameter and a height with which the insertion part 113 inserted into the skull 1 is easily mounted and the fixing protrusion 117 and the first fastening hole 110b are optimally formed, to thereby make it possible to secure a desired fixing force and stability.

The fixing protrusion 117 may satisfy Expression 4.

0.3≤D₄/L₃≤5 Expression 4

Here, D₄ represents an outer diameter of the fixing projection 117, and L₃ represents a height of the fixing projection 117.

In a case where D₄/L₃ is smaller than 0.3, since the height of the fixing protrusion 117 becomes excessively high, the fixing protrusion 117 may be easily damaged when combined with the main body fixing part 150, and even in a fastened state, a part of the main body fixing part 150 may float upward, so that the movable part 130 is not fixed. Further, in a case where D₄/L₃ is greater than 5, since the height of the fixing protrusion 117 becomes excessively low, the fixing function of the fixing protrusion 117 may be insufficient. Accordingly, in a case where the condition range of Expression 4 is satisfied, it is possible to prevent the support part 115 from being rotated or being separated after being fastened to the main body fixing part 150, to thereby maintain the fixing force as time passes.

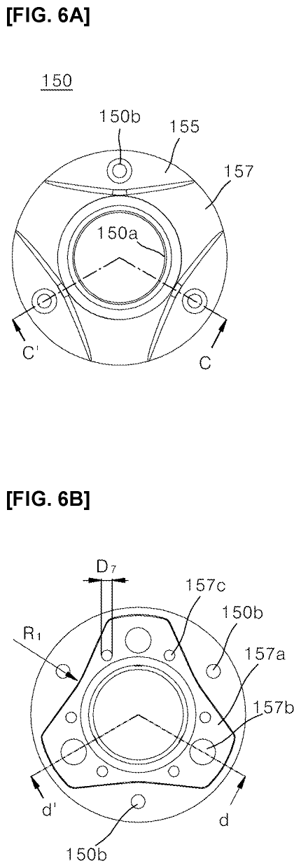

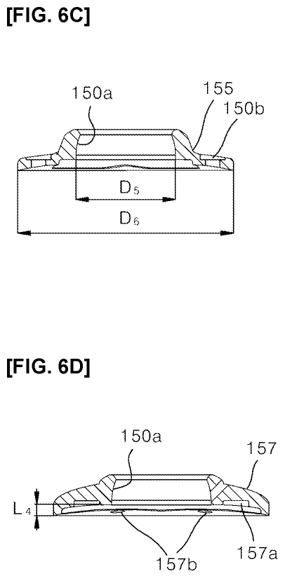

FIGS. 6(a) to 6(d) are a plan view, a bottom view, a c-c′ sectional view, and a d-d′ sectional view showing a main body fixing part of the drug injection device according to the embodiment of the present invention.

Referring to FIGS. 6(a) to 6(d), the main body fixing part 150 may face the main body 110 and be fixedly coupled thereto. The main body fixing part 150 may have the fixing hole 150a formed at a central part thereof for fixing the outside of the movable part 130 so that the movable part 130 is not moved. Further, the main body fixing part 150 may have at least one second fastening hole 150b formed at an edge side thereof, in which the fastening member 170 is fixedly inserted from above.

FIG. 6(c) is a side sectional view taken along c-c′ in FIG. 6(a) that is the plan view of the main body fixing part 150. The fixing hole 150a is formed at the central part of the main body fixing part 150, and the second fastening hole 150b is formed in the mounting part 155 positioned to face the main body 110, in which the fastening member 170 is coupled.

FIG. 6(d) is a side cross-sectional view taken along d-d′ in FIG. 6(b) that is the bottom view of the main body fixing part 150. The main body fixing part 150 having the fixing hole 150a formed at the central part thereof may include the identification part 157 for identifying an implantation position when viewed from the outside. The identification part 157 has a convexly protruding shape compared with the mounting part 155, and the head accommodating groove 157b that faces the first fastening hole 110b of the main body 110 may be formed on its bottom surface. In the head accommodating groove 157b, the head part 175 of the fastening member 170 mounted in the first fastening hole 110b may be located.

The fixing hole 150a and the main body fixing part 150 may satisfy at least one of Expression 5 and a condition that the height of the main body fixing part 150 is in a range of 1 mm to 9 mm.

1≤D₆/D₅≤7.5 [Expression 5]

Here, D₅ represents an outer diameter of the fixing hole 150a, and D₆ represents an outer diameter of the main body fixing part 150.

In a case where D₆/D₅ is smaller than 1, since the fixing hole 150a at the center of the main body fixing part 150 becomes excessively large and a portion exposed outside increases, infection problems may occur, and in a case where D₆/D₅ is greater than 7.5, since the fixing hole 150a at the center of the fixing part 150 becomes excessively small, the insertion of at least one of the sealing part 160 and the movable part 130 into the fixing hole 150a may be difficult. Accordingly, in a case where the condition range of Expression 5 is satisfied, it is possible to easily maintain the fixing force in a state of being coupled with the main body 110, and since the sealing part 160 and the movable part 130 are inserted into the fixing hole 150a, it is possible to prevent infection of contaminants from the outside.

In addition, in a case where the height of the main body fixing part 150 deviates from the range of 1 mm to 9 mm, since the height is higher than the height of the scalp 3 of the patient P to protrude in appearance after implantation, it may cause discomfort to the daily life of the patient P. Accordingly, in a case where the height of the main body fixing part 150 satisfies the range of 1 mm to 9 mm, there is no significant difference in appearance after implantation, the discomfort to the patient P can be reduced, damage caused by external shocks can be reduced, so that the patient P can maintain a smooth daily life.

Referring to FIGS. 6(a) and 6(b), the main body fixing part 150 may include the identification part 157, the fixing hole 150a, the second fastening hole 150b, and the support coupling groove 157a. The identification part 157 that is a curved surface protruding upwards enables identification of the implantation position of the drug injection device 100. The fixing hole 150a may fix the outer surface of the movable part 130 so that the movable part 130 does not move at a central part thereof. At least one second fastening hole 150b formed at the edge side of the main body fixing part 150, in which the fastening member 170 is fixedly inserted from above, may be provided. The support coupling groove 157a is formed to have a curved lower part that is concave in shape and matches the shape of the support part 115 of the main body 10 for easy coupling. Further, the support coupling groove 157a includes at least one protrusion accommodating groove 157c coupled to the fixing protrusion 117 therein, and the support coupling groove 157a may satisfy Expression 6.

5<R₁≤15 [mm] [Expression 6]

Here, R₁ represents a radius of the support coupling groove 157a.

In a case where R₁ is smaller than 5, since it is difficult to accommodate the support part 115 when coupled with the main body 110, the fixing force may be weakened, and in a case where R₁ is greater than 15, since an extra space is generated after the main body 110 is accommodated, the coupling and fixing force with the main body 110 may be weakened. Accordingly, in a case where the condition range of Expression 6 is satisfied, the support coupling groove 157a can have a certain area generated when a circle is divided into three equal parts, to thereby match the shape of the support part 115, and thus, it is possible to maintain the fixing force without separation even in the case of coupling for a long period of time.

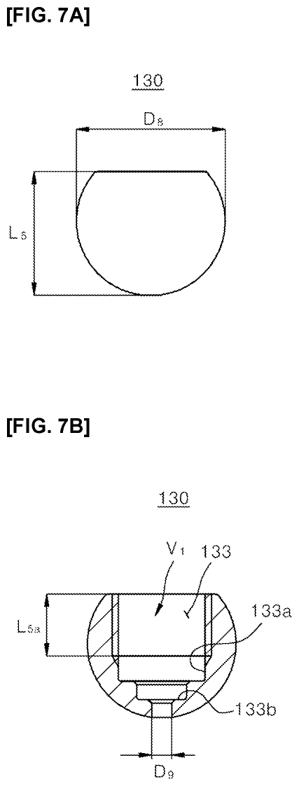

FIGS. 7(a) and 7(b) are a side view and a side sectional view showing a movable part of the drug injection device according to the embodiment of the present invention.

Referring to FIGS. 7(a) and 7(b), the movable part 130 is located between the main body 110 and the main body fixing part 150, and the drug injection hole 133 is formed at the central part thereof to enable the position adjustment to the target point 7. The movable part 130 may include the movable part 130 and the drug injection hole 133. The movable part 130 is located between the movable accommodating groove 110a and the fixing hole 150a, and has an appearance of a curved surface to enable the position adjustment. The drug injection hole 133 may be formed as an internal hole through which the drug is injected into the skull 1.

The movable part 130 may satisfy Expression 7.

0.4≤D₈/L₅≤20 [Expression 7]

Here, D₈ represents an outer diameter of the movable part 130, and L₅ is a height of the movable part 130.

In a case where D₈/L₅ is smaller than 0.4, since the height of the movable part 130 becomes excessively high, there is a limit to providing an internal space for insertion of the sealing part 160 and the rotation may be difficult, and in a case where D₈/L₅ is greater than 20, since the outer diameter of the movable part 130 becomes excessively high, it may be difficult to mount the main body fixing part 150 to the upper part thereof. Further, the brain parenchyma 5 may be pressed, which may cause discomfort and side effects to the patient P after the treatment. Accordingly, in a case where the condition range of Expression 7 is satisfied, the movable part 130 having a spherical shape can be stably positioned between the main body 110 and the main body fixing part 150 to rotate in the direction of the target point 7.

The drug injection hole 133 may include the first accommodating part 133a that accommodates the sealing part 160 that is located in the internal hole of the movable part 130, and the second accommodating part 133b that is located at a lower end part of the inside of the movable part 130 to be connected to the first accommodating part 133a and accommodates an injection needle to be inserted during drug injection. The first accommodating part 133a may be formed with a screw-thread from the start of the internal hole of the movable part 130 to the point at which the sealing part 160 is accommodated.

Each of the first accommodating part 133a and the second accommodating part 133b may satisfy at least one of Expressions 8 and 9.

55≤V₁≤80 [mm³] [Expression 8]

0.5≤D₉≤2 [mm] [Expression 9]

Here, V₁ represents a volume of an inner space of the first accommodating part 133a, and D₉ represents an inner diameter of the second accommodating part 133b.

In a case where V₁ is smaller than 55, since the volume of the inner space of the first accommodating part 133a becomes larger than the inner diameter of the second accommodating part 133b, it may be difficult to find and insert the second accommodating part 133b in insertion of the guide tube 140, and in a case where V₁ is greater than 80, since the inner diameter of the second accommodating part 133b becomes larger than the volume of the internal space of the first accommodating part 133a, it may be difficult to position the guide tube 140 in insertion of the guide tube 140, and the fixing force may decrease over time after the implantation. Accordingly, in a case where the condition range of Expression 8 is satisfied, the sealing part 160 can be stably accommodated in the first accommodating part 133a to block contaminants from the outside, thereby protecting the brain parenchyma 5. Further, since a space for storing the drug can be secured in the first accommodating part 133a, a drug requiring repeated administration in a certain amount, such as a therapeutic agent for dementia or brain disease-related pain, accumulates therein to be administered to the target point 7 while passing through the guide tube 140. Accordingly, the first and second accommodating parts 133a and 133b of the movable part 130 satisfying the condition range of Expression 8 can improve the repeated administration function of the drug injection device 100.

On the other hand, in a case where D₉ is smaller than 0.5, it may be difficult to insert the guide tube 140 into the second accommodating part 133b, and in a case where D₉ is greater than 2, since the inner diameter of the second accommodating part 133b is larger than the outer diameter of the guide tube 140, a gap that is an extra space may be generated. In a case where the drug is injected into the gap, the drug is administered to a part of the brain parenchyma 5 other than the target point 7, which may cause side effects. Accordingly, in a case where the condition range of Expression 9 is satisfied, the inner diameter of the second accommodating part 133b can be suitably coupled to the outer diameter of the guide tube 140, and thus, the drug can be precisely and accurately administered to the target point 7 during drug injection.

The first accommodating part 133a may be formed, at its side surface, with a screw-thread from the start of the internal hole of the movable part 130 to a point at which the sealing part 160 is accommodated.

Here, a height of the screw-thread may satisfy Expression 10.

0.2≤L₅ₐ/L₅≤0.7 [Expression 10]

Here, L₅ represents a total height of the first accommodating part 133a and the second accommodating part 133b, and L₅ₐ represents the height of the screw-thread.

In a case where L₅ₐ/L₅ is smaller than 0.2, since the height of the screw-thread is excessively small, it may be difficult to fasten the movable lid part 180, and in a case where L₅ₐ/L₅ is greater than 0.7, since the height of the screw-thread occupies most of the total height of the first and second accommodating parts 133a and 133b, it may be difficult to accommodate the sealing part 160 in the second accommodating part 133b, and even in a case where it is accommodated, the sealing part 160 may be damaged by the screw-thread. Accordingly, in a case where the condition range of Expression 10 is satisfied, the total height of the first and second accommodating parts 133a and 133b can stably accommodate the sealing part 160 and the guide tube 140, and the height of the screw-thread is suitable for fastening the movable lid part 180, to thereby make it possible to block contaminants from the outside.

FIGS. 8(a) to 8(c) are a plan view, a side view, and a side sectional view showing a movable lid part of the drug injection device according to the embodiment of the present invention.

Referring to FIGS. 8(a) to 8(c), the movable lid part 180 may include the needle accommodating part 180a, the position indicating part 185 and the movable fastening part 183. The needle accommodating part 180a is a hole formed at the central part of the movable lid part 180, into which the injection needle 191 is inserted, and may have a funnel shape that narrows toward the inside so that the injection needle 191 can be inserted into the center no matter which direction the injection needle 191 is inserted. The position indicating part 185 is positioned above the movable part 130, and is formed in a hexagonal shape, in which a corner portion of the hexagon may be molded. However, the present invention is not limited thereto, and various shapes capable of performing position indication may be used. The position indicating part 185 may have an identification function for finding a drug injection position by causing an operator's hand to contact the needle accommodating part 180a in repeatedly administering the drug through the drug injection device 100. The movable fastening part 183 is connected to the position indicating part 185, and is formed with a screw-thread on the outside thereof to be screw-coupled with the drug injection hole 133.

The sealing part 160 and the movable lid part 180 may satisfy at least one of Expressions 11 and 12.

1<D₁₁/D₁₀≤7.2 [Expression 11]

0.4≤L₇/L₆≤20 [Expression 12]

Here, D₁₀ represents an outer diameter of the sealing part 160, D₁₁ represents an outer diameter of the movable lid part 180, L₆ represents a height of the sealing part 160, and L₇ represents a height of the movable lid part 180.

In a case where D₁₁/D₁₀ is smaller than 1, since the outer diameter of the sealing part 160 becomes excessively large, it may push the movable lid part 180 that blocks its upper part, so that foreign substances from the outside may penetrate between the gap generated by the push, which may cause side effects. In a case where D₁₁/D₁₀ is greater than 7.2, since the sealing part 160 is excessively small, the sealing part 160 may not be fixed in the movable part 130, and thus, it may be difficult to pass the injection needle 191 inserted through the movable lid part 180, through the part 160. Accordingly, in a case where the condition range of Expression 11 is satisfied, the movable lid part 180 can always cover the sealing part 160, to thereby prevent foreign substances from penetrating from the outside.

On the other hand, in a case where L₇/L₆ is smaller than 0.4, since the height of the sealing part 160 is excessively large, it may be difficult for the movable lid part 180 to be fastened to the inside of the movable part 130, and in a case where L₇/L₆ is greater than 20, since the height of the movable lid part 180 becomes excessively large, it may protrude above the scalp 3 out of the fixing hole 150a of the main body fixing part 150 after being fastened with the movable part 130. Such a state may cause the patient P to feel uncomfortable in daily life after implantation of the drug injection device 100, and if worse, may cause side effects such as pain-inducing symptoms. Accordingly, in a case where the condition range of Expression 12 is satisfied, the sealing part 160 can be suitably accommodated inside the movable part 130, and the upper part of the sealing part 160 can be suitably covered by the movable lid part 180, to thereby block foreign substances and contamination from the outside, which results in suitability and stability of implantation. Further, the sealing part 160 and the movable lid part 180 having appropriate heights are located between the skull 1 and the scalp 3 without protruding outside, so that the patient P can smoothly conduct daily life after implantation.

The needle accommodating part 180a may satisfy Expression 13.

A₁≥A₂ [Expression 13]

Here, A₁ represents an angle of an upper end part of the needle accommodating part 180a, which is an angle on the left and right with reference to a central axis, and A₂ represents an angle of a middle part of the needle accommodating part 180a.

In a case where the condition range of Expression 13 is not satisfied, since the angle of the middle part becomes sharply large when the injection needle 191 passes through the upper end part of the needle accommodating part 180a, a path direction of the injection needle 191 may be changed, so that the injection needle 191 may be broken or not easily inserted. In a case where the condition range of Expression 13 is satisfied, when the injection needle 191 is in contact with the upper end part of the needle accommodating part 180a from the outside, even though the injection needle 191 is inserted in an arbitrary direction, the injection needle 191 may be finally inserted in the direction of the desired target point 7 through the lower end part of the needle accommodating part 180a.

The position indicating part 185 may satisfy Expression 14.

0.4≤L₇ₐ/L₇<1 [Expression 14]

Here, L₇ represents a total height of the movable lid part 180, and L₇ₐ represents a height of the position indicating part 185.

In a case where L₇ₐ/L₇ is smaller than 0.4, since the movable fastening part 183 occupies most of the movable lid part 180, the fastening with the movable part 130 may be easy, but it may be difficult to separate the movable lid part 180 from the movable part 130 in the process of separating and removal after mounting, and in a case where L₇ₐ/L₇ is greater than 1, since the movable fastening part 183 occupies a low proportion of the movable lid part 180, it may be difficult to fasten to the movable part 130. Accordingly, in a case where the condition range of Expression 14 is satisfied, the movable lid part 180 can be stably fastened to the movable part 130, to thereby make it possible to block contaminants from the outside, and to easily perform the mounting and separation.

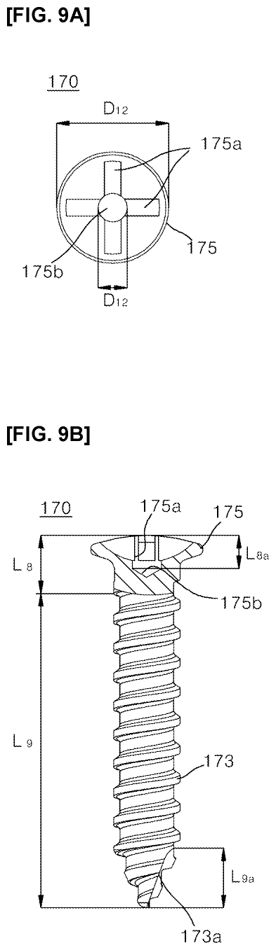

FIGS. 9(a) and 9(b) are a plan view and a side view showing a fastening member of the drug injection device according to the embodiment of the present invention.

Referring to FIGS. 9(a) and 9(b), the fastening member 170 may include at least two members for fastening the upper part of the main body 110 and the upper part of the main body fixing part 150 to the skull 1. The fastening member 170 may include the body part 173 that is fixedly implanted in the skull and the head part 175 that is connected to the body part 173.

The body part 173 and the head part 175 may satisfy at least one of Expressions 15 and 16.

1<D₁₂/D₁₁≤50 [Expression 15]

1<L₉/L₈≤100 [Expression 16]

Here, D₁₁ represents an outer diameter of the body part 173, D₁₂ represents an outer diameter of the head part 175, L₉ represents a length of the body part 173, and L₈ represents a length of the head part 175.

In a case where D₁₂/D₁₁ is smaller than 1, since the diameter of the body 173 becomes larger than the diameter of the head 175, it may fall into the hole to be fastened without being mounted after fastening, and in a case where D₁₂/D₁₁ is greater than 50, since the head part 175 becomes excessively large, the fastening may be difficult due to interference by the main body fixing part 150, and the strength of the body part 173 may become relatively weak, so that the fixing force may be lowered and stable fastening may not be secured. Accordingly, in a case the condition range of Expression 15 is satisfied, the fastening member 170 can fix the main body 110 and the main body fixing part 150, and can improve the implantation stability and fixing force of the drug injection device 100.

On the other hand, in a case where L₉/L₈ is smaller than 1, since the length of the head 175 becomes excessively long, it may be difficult to fixedly support the implanted body part 173, and in a case where L₉/L₈ is greater than 100, since the body part 173 becomes excessively long, the body part 173 may deviate from the skull 1 to press the brain parenchyma 5, which may cause side effects after implantation. Accordingly, in a case where the condition range of Expression 16 is satisfied, the fastening member 170 can be stably implanted in the skull 1 at an appropriate ratio between the head part 175 and the body part 173 to fix the drug injection device 100.

The head part 175 may include the horizontal groove 175b formed at a central part thereof to be horizontally fastened to a fastening tool in fastening, and the identification groove 175a formed to be identifiable in a cross shape with reference to the horizontal groove 175b.

The horizontal groove 175b and the identification groove 175a may satisfy Expression 17.

0.06≤D₁₃/L₈ₐ≤12 [Expression 17]

Here, L₈ₐ represents a depth of the identification groove 175a, and D₁₃ represents an outer diameter of the horizontal groove 175b.

In a case where D₁₃/L₈ₐ is smaller than 0.06, since the length of the identification groove 175a becomes excessively long, the function of the horizontal groove 175b may be lost, and in a case where D₁₃/L₈ₐ is greater than 12, since the outer diameter of the horizontal groove 175b becomes excessive large, the identification function of the identification groove 175a may be reduced. Accordingly, in a case where the condition range of Expression 17 is satisfied, the head part 175 can have the identification function of the identification groove 175a and the function of allowing the fastening member 170 of the horizontal groove 175b to be fastened horizontally.

The body part 173 is fixedly implanted in the skull, has a screw-thread tapered to the end part thereof, and may be formed with at least one pressure reducing groove 173a that satisfies the following Expression 18.

1<L₉/L₉ₐ≤50 [Expression 18]

Here, L₉ represents a length of the body part 173, and L₉ₐ represents a length of a portion in which the pressure reducing groove 173a is formed.

In a case where L₉/L₉a is smaller than 1, since the pressure reducing groove 173a is relatively longer than the length of the body part 173, the function of the fastening member 170 may be lost and the fixing force may be weakened, and in a case where L₉/L₉a is greater than 50, since the fastening member 170 has almost no pressure reducing groove 173a, it may increase the pressure on the bone tissue during implantation. In this case, implantation for a long time may damage bone tissues to cause inflammation, which may result in severe adverse effects of the implantation. Accordingly, in a case where the condition range of Expression 18 is satisfied, when the fastening member 170 having the pressure reducing groove 173a is implanted in the bone, it is possible to reduce the pressure that damages bone tissues, thereby minimizing bone damage.

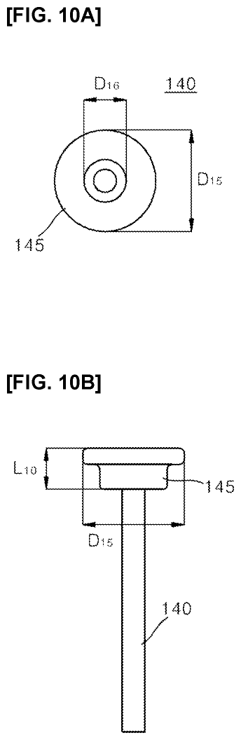

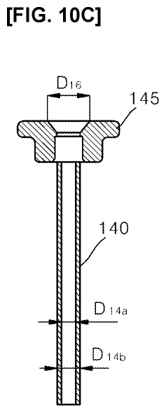

FIGS. 10(a) to 10(c) are a plan view, a side view, and a side sectional view showing a guide tube of the drug injection device according to the embodiment of the present invention.

Referring to FIGS. 10(a) to 10(c), the guide tube 140 may guide an injection needle for injecting a drug to the target point 7 in the target direction. Further, the guide tube 140 may have a smooth surface to minimize damage when passing through the brain parenchyma 5.

Here, the guide tube 140 may have a length ranging from 10 mm to 300 mm, and may satisfy Expression 19.

1<D_{14b}/D₁₄ₐ≤3.5 [Expression 19]

Here, D₁₄ₐ represents an inner diameter of the guide tube 140, and D_{14b} represents an outer diameter of the guide tube 140.

In a case where D_{14b}/D₁₄ₐ is smaller than 1, the inside of the guide tube 140 may be torn or damaged when the injection needle 191 passes therethrough due to the thin thickness of the guide tube 140 in insertion of the injection needle 191. Further, in a case where the guide tube 140 is inserted into the brain parenchyma 5, the strength is too weak to cause adhesion to the brain parenchyma 5, which results in side effects, or the function of accommodating and guiding the injection needle 191 may be lost. Further, in a case where D_{14b}/D₁₄ₐ is greater than 3.5, since the thickness of the guide tube 140 becomes excessively thick, it may be difficult to insert the injection needle 191 therein. In addition, since the insertion strength according to the thickness becomes excessively strong, the guide tube 140 may cause damage when inserted into the brain parenchyma 5. Accordingly, in a case where the condition range of Expression 19 is satisfied, the guide tube 140 can be inserted first to the brain parenchyma 5 to minimize damage, and then, can guide the injection needle 191 in the direction of the target point 7 when the injection needle 191 is inserted inside the guide tube 140.

On the other hand, the guide tube 140 may further include the tube connection part 145. The tube connection part 145 is mounted at the lower end part of the drug injection hole 133 to connect the movable part 130 and the guide tube 140, and may be formed with the insertion guide hole 145a connected to the inside of the guide tube 140. The insertion guide hole 145a may have a funnel shape that becomes narrower downward, but the present invention is not limited thereto, and various shapes capable of providing guidance in the central direction may be used. Accordingly, even in a case where the injection needle 191 deviates in a wrong direction other than the center when passing through the needle accommodating part 180a, the insertion guide hole 145a can guide the injection needle 191 together with the needle accommodating part 180a in the central direction to be accurately inserted into the target point 7.

The tube connection part 145 may satisfy at least one of Expressions 20 and 21.

1<D₁₅/D₁₆≤20 [Expression 20]

0.5≤L₁₀≤1.5 [Expression 21]

Here, D₁₅ represents an outer diameter of the tube connection part 145, D₁₆ represents an inner diameter of the tube connection part 145, and L₁₀ represents a height of the tube connection part 145.

In a case where D₁₅/D₁₆ is smaller than 1, since the inner diameter of the tube connection part 145 becomes excessively large and the thickness of the tube connection part 145 becomes excessively thin, the tube connection part 145 may be torn or damaged when the injection needle 191 is inserted therein, and in a case where D₁₅/D₁₆ is greater than 20, since the thickness of the tube connection part 145 becomes excessively large and the internal space becomes excessively small, the insertion and separation of the needle 191 may be difficult. Accordingly, in a case where the condition range of Expression 20 is satisfied, the tube connection part 145 can be stably supported and fixed to the lower end part of the movable part 130 without separation. Further, the tube connection part 145 can guide the injection needle 191 in the direction of the target point 7 as the injection needle 191 passes through the inside of the tube connection part 145.

On the other hand, in a case where L₁₀ is smaller than 0.5, since the height of the tube connection part 145 is excessively low, the fixing and supporting force may be weakened, so that the tube connection part 145 may be separated to the brain parenchyma 5, resulting in side effects, and in a case where L₁₀ is greater than 1.5, since the height of the tube connection part 145 is excessively high, it may be difficult for the tube connection part 145 to be accommodated together with the sealing part 160 inside the movable part 130. Accordingly, in a case where the condition range of Expression 21 is satisfied, the tube connection part 145 can be mounted and supported inside the movable part 130 with a stable fixing force, and it is possible to block contaminants from the outside by mounting the sealing part 160 at the upper part thereof.

At least one drug injection hole 140a may be formed in the guide tube 140 so that the drug can flow without accumulation on one side or both sides of the guide tube 140. Further, the guide tube 140 may have a shape in which the inner and outer diameters of the end parts are formed in a curved shape to minimize tissue damage when inserted into the skull 1.

In the drug injection device 100, at least one of the main body 110, the main body fixing part 150, the movable part 130, the movable lid part 180, and the guide tube 140 may be made of polyether ether ketone (PEEK).

The fastening member 170 may be made of a titanium material. However, the present invention is not limited thereto, and the drug injection device 100 and the fastening member 170 may employ various materials as long as the materials have body implantation suitability and stability, suitable hardness, and resistance to external contamination.

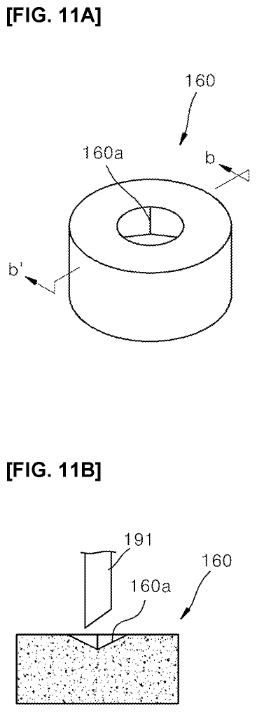

FIGS. 11(a) and 11(b) are a perspective view and a side sectional view showing a first modified example of a sealing part of the drug injection device according to the embodiment of the present invention.

Referring to FIGS. 11(a) and 11(b), a central guide groove 160a may be formed at a central part of the sealing part 160. The central guide groove 160a may guide the injection needle 191 toward the center even in a case where the needle 191 is inserted in a different direction. In the central guide groove 160a, at least three points may be cut with reference to a central point thereof. Specifically, its cutting line may have a ‘Y’ or ‘X’ shape, but is not limited thereto, and may have various shapes with reference to the central point.

Accordingly, the central guide groove 160a has an effect of preventing damage to other points of the sealing part 160 and continuously guiding the injection needle 191 toward the guide tube 140.

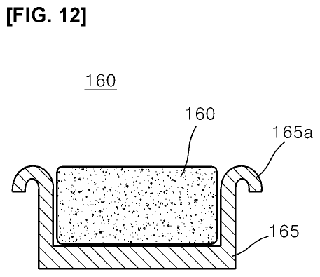

FIG. 12 is a side sectional view showing a second modified example in which a filter is integrated into the sealing part of the drug injection device according to the embodiment of the present invention.

Referring to FIG. 12, the sealing part 160 may further include a sealing filter membrane 165 that prevents foreign substances from being introduced therein. The sealing filter membrane 165 may be formed in a one-piece type in which the filter and the sealing part 160 are integrated, so that mounting and exchange thereof can be easily performed at once. The sealing filter membrane 165 may have a hinge part 165a that is folded outward at its upper part. In a case where the sealing filter membrane 165 is exchanged, the sealing filter membrane 165 can be easily held and taken out using the hinge part 165a. Accordingly, the sealing filter membrane 165 can prevent separation when exchanging the consumable sealing part 160, and can prevent contamination of the sealing part 160 over time, to thereby reduce the side effects of infection.

On the other hand, the sealing part 160 may include at least one of a first mixture and a second mixture.

The first mixture may contain 55 to 60% by weight of siloxane, silicone, dimethyl and vinyl groups, 25 to 30% by weight of hydrolysis products of silane amine, 1.1.1-trimethylamine trimethylsilyl and silica, and 5 to 7% by weight of siloxane silicone, dimethyl and methylvinyl.

The second mixture may contain 55 to 60% by weight of siloxane, silicone, dimethyl and vinyl groups, 25 to 30% by weight of hydrolysis products of silane amine, 1.1.1-trimethylaminetrimethylsilyl and silica, 5 to 7% by weight of siloxane silicone, dimethyl and methylvinyl, and 1 to 5% by weight of silicon siloxane, dimethyl and methyl hydrogen.

Here, in the sealing part 160, the first mixture and the second mixture may satisfy Expression 22.

1≤M₂/M₁≤2 [Expression 22]

Here, M₁ represents the first mixture, and M₂ represents the second mixture.

In a case where M₂/M₁ is smaller than 1, the density of the sealing part 160 may be weakened, and thus, separation may occur over time, and hardening or burning may occur in blending the mixtures. Further, lots of bubbles may be generated on the surface of the sealing part 160 and the injection needle 191 may be inserted with silicone particles to be penetrated into the brain parenchyma 5, which may cause side effects after implantation. In addition, in a case where M₂/M₁ is greater than 2, since the density of the sealing part 160 becomes excessively strong, it may be difficult to insert the injection needle 191, or the needle 191 may be broken. Accordingly, in a case where the condition range of Expression 22 is satisfied, the sealing part 160 can have the most appropriate blending ratio, so that the injection needle 191 can be easily inserted into the surface thereof, and the bonding between the silicone particles becomes strong to minimize bubbles on the surface of the sealing part 160.

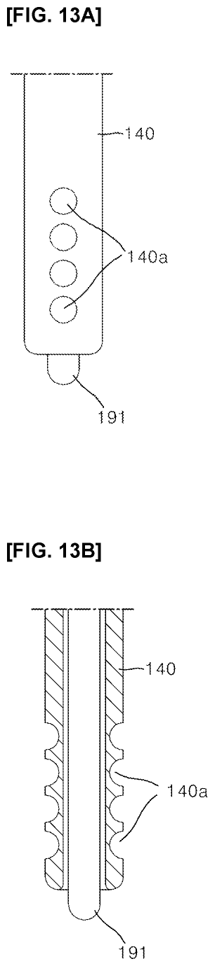

FIG. 13 is a side sectional view showing a state where an end part of the guide tube of the drug injection device according to the embodiment of the present invention is deformed.

Referring to FIG. 13, at least one drug injection hole 140a may be formed on one or both sides of the guide tube 140 so that the drug, that is, the therapeutic agent 195 flows without being accumulated thereon. The drug injection hole 140a has a circular shape, and may be formed in various shapes. Further, since the inner diameter and outer diameter of the end part of the guide tube 140 are formed in a curved line, it is possible to minimize tissue damage when passing through the brain parenchyma 5 together with the injection needle 191.

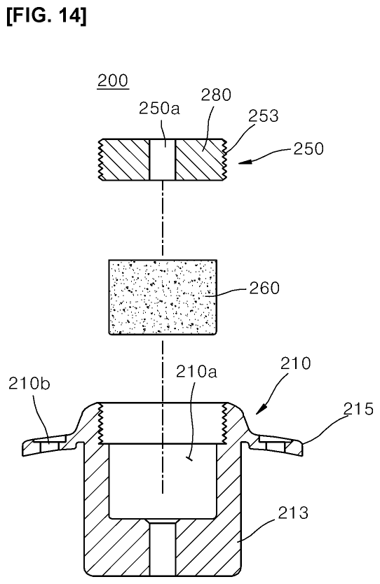

FIG. 14 is a side sectional view showing a drug injection device according to a second embodiment of the present invention.

Referring to FIG. 14, a drug injection device 200 according to the second embodiment of the present invention may include a main body 210, a main body fixing part 250, and a sealing part 260. The main body 210 is formed with a sealing accommodating hole 210a at a central part thereof, and is formed with a needle insertion hole 210b that is connected to the sealing accommodating hole 210a, into which the needle 191 is inserted. Further, the main body 210 includes a support part 215 at an upper part thereof, and at least two fastening holes 210b may be formed in the support part 215. The sealing accommodating hole 210a has a rectangular space that accommodates the sealing part 260, and the injection needle 191 can pass through the space. The main body fixing part 250 has a fixing hole 250a formed at a central part thereof, and a body sealing part 253 formed with a screw-thread to be screw-coupled to the inside of the main body 210 on a side surface thereof. Compared with the drug injection device 100 of FIG. 1, the injection needle 191 may be inserted in only one direction through the drug injection device 200. The injection needle 191 may be inserted into the sealing accommodating hole 210a passing through the fixing hole 250a and the sealing part 260. Since the drug injection device 200 according to the second embodiment of the present invention has such a simple structure, and thus, it is possible to reduce time for mounting, separation and removal, thereby easily exchanging the sealing part 260.

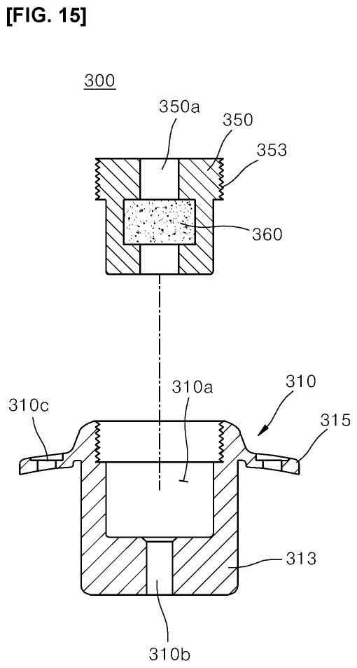

FIG. 15 is a side sectional view showing a drug injection device according to a third embodiment of the present invention.

Referring to FIG. 15, a drug injection device 300 according to the third embodiment of the present invention may include a main body 310, a main body fixing part 350, and a sealing part 360. The main body 310 is formed with a sealing accommodating hole 310a at a central part thereof, and is connected to the sealing accommodating hole 310a to have a needle insertion hole 310b into which the injection needle 191 is inserted. Further, the main body 310 includes a support part 315 at an upper part thereof, and at least two fastening holes 310b may be formed in the support part 315. The sealing accommodating hole 310a has a rectangular space, and the injection needle 191 can pass through the space. Compared with FIG. 14, the fixing hole 350a is formed at the center of the main body fixing part 350, and a sealing part 360 is accommodated in the fixing hole 350a. A body sealing part 353 formed with a screw-thread to be screw-coupled to the inside of the body 310 may be formed on a side surface of the main body fixing part 350. Compared with the drug injection device 100 of FIG. 1, the injection needle 191 may be inserted in only one direction through the drug injection device 300. The injection needle 191 may be inserted into the sealing accommodating hole 310a passing through the sealing part 360 and the fixing hole 350a. Since the drug injection device 300 according to the third embodiment of the present invention has such a simple structure, and thus, it is possible to easily exchange the sealing part 360 by only fastening and separating the main body fixing part 350. Further, it is possible to reduce time for mounting, separation and removal.

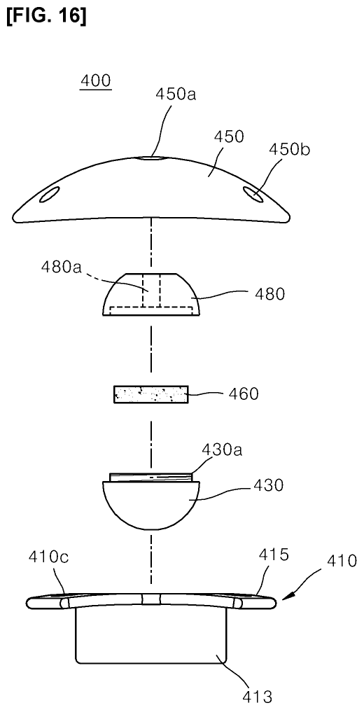

FIG. 16 is a front view showing a drug injection device according to a fourth embodiment of the present invention.

Referring to FIG. 16, a drug injection device 400 according to the fourth embodiment of the present invention may include a main body 410, a movable part 430, a main body fixing part 450, a sealing part 460, and a movable lid part 480. The main body 410 may have a movable part accommodating hole 410a for accommodating the movable part 430 at a central part thereof. Further, the main body 410 may be formed with a needle insertion hole 410b connected to the movable part accommodating hole 410a at a lower end part thereof, into which the needle 191 is inserted. In addition, the main body 410 may include an insertion part 413 inserted into the skull 1 and a support part 415 mounted above the skull 1. At least two fastening holes 410c may be formed in the support part 415. The drug injection device 400 according to the present embodiment is similar to the drug injection device 100 of FIG. 1 in that the movable part 430 can be rotated left and right with reference to the central axis CL, so that the injection needle 191 can be guided in the direction of the target point 7, and the internal hole is formed therein. Accordingly, since the sealing part 460 is accommodated between the movable part 430 and the movable lid part 480 to be doubly blocked from the outside, it is possible to prevent infection from external contaminants. Further, the drug injection device 400 according to the present embodiment is similar to the drug injection device 100 of FIG. 1 in that the main body fixing part 450 is formed with the fixing hole 450a into which the drug is injected at the central part thereof.

On the other hand, the drug injection device 400 according to the present embodiment is different from the drug injection device 100 of FIG. 1 in that the movable part 430 is formed in a hemispherical shape, and can be rotated left and right with reference to the central axis CL with a complete spherical shape after being coupled to the movable lid part 480. Further, in the main body fixing part 450, the second fastening hole 450b is formed at least two points, which are positioned to face the fastening holes 410c of the main body 410. Accordingly, the main body 410 and the main body fixing part 450 may be coupled at least three points, to thereby fasten the fastening member 170.

The above-described embodiments are merely exemplary, and various modifications and equivalent other embodiments may be made by those skilled in the art to which the present invention belongs. Accordingly, the true technical protection scope of the present invention should be determined by the technical idea of the invention described in claims.

1. A drug injection device that is fixedly implanted between the skull and the scalp of a patient to inject a drug, comprising:
a main body that is fixedly implanted in contact with the skull and has a movable part accommodating hole as an internal hole;
a main body fixing part that has an internal hole to be adjacent to the main body and is fixedly coupled to the main body to face the main body;
a movable part that is positioned between the main body and the main body fixing part, has a drug injection hole at a central part thereof, and is able to change its direction to a target point;
a sealing part that is configured to seal the drug injection hole of the movable part, into which an injection needle for injecting a drug is inserted;
a movable lid part that is positioned above the sealing part to close and fix the movable part, prevents the sealing part from being separated and escaping from the outside, and identifies an implantation position of the drug injection device to guide a point for repeated administration; and
at least two fastening members that fasten an upper part of the main body and an upper part of the main body fixing part to the skull,
wherein the main body includes a support part that is positioned above the skull for support, an outer diameter of the support part is smaller than an outer diameter of the main body fixing part and is greater than an outer diameter of the movable part, and the main body and the main body fixing part are adjacently coupled to each other to accommodate and fix the movable part in the internal hole of the main body.

2. The drug injection device according to claim 1,
wherein the main body further includes an insertion part that is inserted into the skull, and satisfies at least one of the following Conditional Expressions 1 and 2:
1<D₁/D₂≤3 <Conditional Expression 1>
2≤L₁≤7 [mm] <Conditional Expression 2>
where D1 represents an outer diameter of the insertion part, D2 represents an inner diameter of the movable part accommodating hole, and L1 represents an insertion height of the insertion part.

3. The drug injection device according to claim 1,
wherein the support part includes
at least one first fastening hole that is positioned at the upper part of the main body, in which the fastening member is fixedly inserted; and
at least one fixing protrusion that is fixedly coupled to the main body fixing part, and
satisfies the following Conditional Expression 3:
12≤D₃/L₂≤60 <Conditional Expression 3>
where D3 represents the outer diameter of the support part, and L2 represents a height of the support part.

4. The drug injection device according to claim 3,
wherein the fixing protrusion satisfies the following Conditional Expression 4:
0.3≤D₄/L₃≤5 <Conditional Expression 4>
where D4 represents an outer diameter of the fixing projection, and L3 represents a height of the fixing projection.

5. The drug injection device according to claim 1,
wherein the main body fixing part has a fixing hole formed at a central part thereof for fixing the outside thereof so that the movable part is not moved, and has at least one second fastening hole formed at an edge side of the main body fixing part, in which the fastening member is fixedly inserted from above, and
the fixing hole and the main body fixing part satisfy at least one of the following Conditional Expression 5 and a condition that the height of the main body fixing part is in a range of 1 mm to 9 mm:
1<D₆/D₅≤7.5 <Conditional Expression 5>
where D5 represents an outer diameter of the fixing hole and D6 represents the outer diameter of the main body fixing part.

6. The drug injection device according to claim 5,
wherein the main body fixing part includes an identification part that has a curved surface that protrudes upwards to be identifiable, and
a support coupling groove that has a curved lower part that is concave in shape and matches the shape of the support part of the main body for easy coupling, and
the support coupling groove is formed with at least one projection accommodating groove and a head accommodating groove that face the main body for coupling, and satisfies the following Conditional Expression 6:
5<R₁≤15 [mm] <Conditional Expression 6>
where R1 represents a radius of the support coupling groove.

7. The drug injection device according to claim 1,
wherein the movable part includes a movable main body part that is positioned in the movable part accommodating hole, and is formed with a curved surface in shape to enable direction change, and the movable main body part is formed with a drug injection hole for guiding the drug to be injected to the target point at a central part thereof.

8. The drug injection device according to claim 1,
wherein the movable part includes a drug injection hole that is an internal hole through which the drug is injected into the skull, and the movable part satisfies the following Conditional Expression 7:
0.4≤D₈/L₅≤20 <Conditional Expression 7>
where D8 represents the outer diameter of the movable part and L5 represents a height of the movable part.

9. The drug injection device according to claim 8,
wherein the movable part is formed in a spherical shape having a sphericity less than 0.03 mm.

10. The drug injection device according to claim 8,
wherein the drug injection hole includes
a first accommodating part that accommodates the sealing part located in the internal hole of the movable part, and
a second accommodating part that is connected to the first accommodating part, is located at a lower end part of the inside of the movable part, and accommodates the injection needle inserted during drug injection, and
each of the first accommodating part and the second accommodating part satisfies at least one of Conditional Expressions 8 and 9:
55≤V₁≤80 [mm³] <Conditional Expression 8>
0.5≤D₉≤2 [mm] <Conditional Expression 9>
where V1 represents the volume of an internal space of the first accommodating part, and D9 represents an inner diameter of the second accommodating part.

11. The drug injection device according to claim 8,
wherein the first accommodating part is formed with a screw-thread from the start of the internal hole of the movable part to a point at which the sealing part is accommodated, and a height of the screw-thread satisfies the following Conditional Expression 10:
0.2≤L₅ₐ/L₅≤0.7 <Conditional Expression 10>
where L5 represents a total height of the first accommodating part and the second accommodating part, and L5a represents a height of the screw-thread.

12. The drug injection device according to claim 1,
wherein the sealing part is configured so that the sealing part and the movable lid part satisfy at least one of the following Conditional Expressions 11 and 12:
1<D₁₁/D₁₀≤7.2 <Conditional Expression 11>
0.4≤L₇/L₆≤20 <Conditional Expression 12>
where D10 represents an outer diameter of the sealing part, D11 represents an outer diameter of the movable lid part, L6 represents a height of the sealing part, and L7 represents a height of the movable lid part.

13. (canceled)

14. The drug injection device according to claim 1,
wherein the movable lid part includes
an injection needle accommodating part that is formed with a hole into which the injection needle is injected at a central part thereof,
a position indicating part that includes the needle accommodating part therein and is positioned outside the movable part, and
a movable fastening member that is connected to the position indicating part, is formed with a screw-thread on the outside thereof, and is screw-coupled with the drug injection hole.

15. The drug injection device according to claim 14,
wherein the injection needle accommodating part satisfies the following Conditional Expression 13:
A₁≥A₂ <Conditional Expression 13>
where A1 represents an angle of an upper end part of the needle accommodating part, which is an angle on the left and right with reference to a central axis, and A2 represents an angle of a middle portion of the needle accommodating part.

16. The drug injection device according to claim 14,
wherein the position indicating part satisfies the following Conditional Expression 14:
0.4≤L₇ₐ/L₇<1 <Conditional Expression 14>
where L7 represents a total height of the movable lid part, and L7a represents a height of the position indicating part.

17. The drug injection device according to claim 1,
wherein the fastening member includes
a body part that is fixedly implanted in the skull, and
a head part that is connected to the body part, and
the body part and the head part satisfy at least one of the following Conditional Expressions 15 and 16:
1<D₁₂/D₁₁≤50 <Conditional Expression 15>
1<L₉/L₈≤100 <Conditional Expression 16>
where D11 represents an outer diameter of the body part, D12 represents an outer diameter of the head part, L9 represents a length of the body part, and L8 represents a length of the head part.

18. The drug injection device according to claim 17,
wherein the head part includes
a horizontal groove that is positioned at a central part thereof to be horizontally coupled to a fastening tool in fastening, and
an identification groove that is formed to be identifiable in a cross shape with reference to the horizontal groove, and
the horizontal groove and the identification groove satisfy the following Conditional Expression 17:
0.06≤D₁₃/L₈ₐ≤12 <Condition 17>
where L8a represents a depth of the identification groove, and D13 represents an outer diameter of the horizontal groove.

19. The drug injection device according to claim 17,
wherein the body part is fixedly implanted in the skull and is formed with a tapered screw-thread to the end thereof, and is formed with at least one pressure reducing groove that satisfies the following Conditional Expression 18:
1<L₉/L₉ₐ≤50 <Conditional Expression 18>
where L9 represents a length of the body part, and L9a represents a length of a portion in which the pressure reducing groove is formed.

20. The drug injection device according to claim 1, further comprising a guide tube that is connected to the movable part and is inserted to a position close to the target point, and guides the injection needle for injecting the drug into the target point in the direction of the target point.

21. The drug injection device according to claim 20,
wherein the guide tube further includes a tube connection part that is mounted on a lower end part of the drug injection hole, connects the movable part and the guide tube, and is formed with a hole connected to the inside of the guide tube.

22. The drug injection device according to claim 20,
wherein the guide tube satisfies the following Conditional Expression 19:
1<D_{14b}/D₁₄ₐ≤3.5 <Conditional Expression 19>
where D14a represents an inner diameter of the guide tube, and D14b represents an outer diameter of the guide tube.

23. (canceled)

24. The drug injection device according to claim 21,
wherein the tube connection part satisfies at least one of the following Conditional Expressions 20 and 21:
1<D₁₅/D₁₆≤20 <Conditional Expression 20>
0.5≤L₁₀≤1.5 [mm] <Conditional Expression 21>
where D15 represents an outer diameter of the tube connection part, D16 represents an inner diameter of the tube connection part, and L10 represents a height of the tube connection part.

25. The drug injection device according to claim 20,
wherein the guide tube is configured so that at least one drug injection hole is formed on one side or both sides thereof so that the drug flows without accumulation, and the inner and outer diameters of the end part of the guide tube are formed in a curved shape to minimize tissue damage when inserted into the skull.

26-31. (canceled)
